SEQUENCE LISTING (3 sequences)

SEQ ID NO: 1 - protein, 9 aa, synthetic construct
  REGION 1..9: note = peptide sequence
FSRSLHSLL

SEQ ID NO: 2 - protein, 7 aa, synthetic construct
  REGION 1..7: note = peptide sequence
CQCRRKN

SEQ ID NO: 3 - protein, 7 aa, synthetic construct
  REGION 1..7: note = peptide sequence
AQARRKN

(19) United States
(12) Patent Application Publication
(10) Pub. No.: US 2023/0320996 A1
(43) Pub. Date: Oct. 12, 2023

(54) NANOPARTICLES AND METHODS OF MANUFACTURE THEREOF

(71) Applicant: Hillstream BioPharma, Inc., Chester, NJ (US)

(72) Inventors: Harpal SINGH, New Delhi (IN); Randy MILBY, Chester, NJ (US); Sachchidanand TIWARI, Bareilly (IN); Anees MOHAMMAD, New Delhi (IN)

(21) Appl. No.: 18/145,350

(22) Filed: Dec. 22, 2022

Related U.S. Application Data
(63) Continuation of application No. PCT/US21/39889, filed on Jun. 30, 2021.
(60) Provisional application No. 63/046,508, filed on Jun. 30, 2020.

(51) Int. Cl. A61K 9/51; A61K 31/35; A61K 38/08; A61K 39/00; A61K 31/337; A61K 31/704; A61K 31/454; A61K 31/505; A61K 31/4745; A61K 48/00; A61K 38/20; A61K 38/19; A61K 38/21

(52) U.S. Cl. CPC A61K 9/5153; A61K 9/5146; A61K 31/35; A61K 38/08; A61K 39/00117; A61K 31/337; A61K 31/704; A61K 31/454; A61K 31/505; A61K 31/4745; A61K 48/0041; A61K 38/2013; A61K 38/191; A61K 38/217; A61K 9/5192

(57) ABSTRACT

Provided herein is a biodegradable polymeric nanoparticle formed of hybrid block copolymers comprising a di-block copolymer methoxy-poly(ethylene glycol)-poly(lactic acid) (m-PEG-PLA) and/or a penta-block copolymer poly(lactic acid)-poly(ethylene glycol)-poly(propylene glycol)-poly(ethylene glycol)-poly(lactic acid) (PLA-PEG-PPG-PEG-PLA). Also provided herein are methods of preparing biodegradable polymeric nanoparticles.

### CROSS REFERENCE TO RELATED APPLICATIONS

This application claims the benefit of U.S. Provisional Patent Application Ser. No. 63/046,508, filed on Jun. 30, 2020, the entire contents of which are incorporated herein by reference in their entirety.

### INCORPORATION BY REFERENCE OF A SEQUENCE LISTING

A sequence listing is provided herein as a text file titled “184470_Seqlisting.txt”, which was created on Jun. 16, 2021 and has a size of 785 bytes. The sequence listing is incorporated herein by reference in its entirety.

### FIELD

The present invention relates to the field of nanotechnology, in particular, to the use and manufacture of biodegradable polymeric nanoparticles for the delivery of therapeutic agents such as salinomycin.

### BACKGROUND

Molecularly targeted therapy has emerged as a promising approach to overcome the lack of specificity of conventional chemotherapeutic agents in the treatment of cancer. Targeted delivery of anticancer drugs would be more effective if the delivery system was able to reach the desired tumor tissues through the penetration of barriers in the body with minimal loss of their volume or activity in the blood circulation and selectively kill tumor cells. This would improve patient survival and quality of life by increasing the intracellular concentration of drugs and reducing dose-limiting toxicities simultaneously. There is a pressing need for a delivery system that can effectively deliver therapeutic agents into the cytosol of cancerous cells.

### SUMMARY

This disclosure provides a composition comprising a biodegradable polymeric nanoparticle formed of hybrid block copolymers comprising a di-block copolymer methoxy-poly(ethylene glycol)-poly(lactic acid) (m-PEG-PLA) and/or a penta-block copolymer poly(lactic acid)-poly(ethylene glycol)-poly(propylene glycol)-poly(ethylene glycol)-poly(lactic acid) (PLA-PEG-PPG-PEG-PLA). In some embodiments, one or both of the di-block copolymer and the penta-block copolymer comprise an average molecular weight of about 5,000 to 30,000 g/mol. In some embodiments, the polymeric nanoparticle has an average diameter of about 40-150 nm.

In some embodiments, the composition is substantially free of emulsifier. In other embodiments, the composition further comprises external emulsifier of about 0.5% to 5% by weight, based on the total weight of the composition.

In some embodiments, the biodegradable polymeric nanoparticle further comprises a therapeutic agent. In some embodiments, the therapeutic agent is associated substantially with the biodegradable polymeric nanoparticle. In some embodiments, the therapeutic agent is selected from a group comprising small organic molecules, nucleic acids, polynucleotides, oligonucleotides, nucleosides, DNA, RNA, amino acids, peptides, proteins, antibiotics, low molecular weight molecules, chemotherapeutics, drugs, metal ions, dyes, radioisotopes, contrast agents and imaging agents. In one embodiment, the antibiotic comprises salinomycin.

In some embodiments, the composition further comprises a stabilizer. In some embodiments, the stabilizer is selected from a group consisting of mannose, beta-lactose, trehalose, sodium cholate, and glucose. In some embodiments, the stabilizer is present in a weight of about 5% to about 50% of the total weight of the polymer.

In some embodiments, the drug is bortezomib.

In some embodiments, the peptide is an anti-cancer peptide. In some embodiments, the anticancer peptide is either FSRSLHSLL (SEQ ID NO: 1) or any polypeptide substantially incorporating the FSRSLHSLL (SEQ ID NO: 1), and the FSRSLHSLL (SEQ ID NO: 1) is in either the D or L-configuration. In some embodiments, the anti-cancer peptide is chemically modified with a hydrophobic polymer. In other embodiments, the anticancer peptide is either FSRSLHSLL (SEQ ID NO: 1) or any polypeptide substantially incorporating the FSRSLHSLL (SEQ ID NO: 1), wherein the FSRSLHSLL (SEQ ID NO: 1) is in either the D or L-configuration, and the hydrophobic polymer is poly(lactic acid).

In some embodiments, the anti-cancer peptide is CQCRRKN (SEQ ID NO: 2), a sequence from the MUC1-CD domain. In other embodiments, the anti-cancer peptide is AQARRKN (SEQ ID NO: 3), a modified sequence from the MUC1-CD domain. In some embodiments, the anticancer peptide is linked to a protein transduction domain. In some embodiments, the protein transduction domain comprises a polyarginine domain.

In some embodiments, the biodegradable polymeric nanoparticle further comprises a chemotherapeutic agent. In some embodiments, the chemotherapeutic is selected from the group consisting of paclitaxel, doxorubicin, pimozide, perimethamine, topoisomerase I inhibitors such as irinotecan, topotecan, indenoisoquinolines, or nor-indenoisoquinolines, HDAC6 inhibitors, PI3Kalpha, beta, gamma or delta inhibitors or PI3-Kdelta/HDAC6 dual inhibitors.

In some embodiments, the therapeutic agent is DNA. In some embodiments, the DNA comprises a polynucleotide encoding a protein comprising the amino acid sequence of TNF, IL-2 or gamma Interferon.

In some embodiments, the peptide comprises the amino acid sequence from one chain of insulin.

In some embodiments, the biodegradable polymeric nanoparticle further comprises a targeting moiety selected from the group consisting of vitamins, small molecule drugs, ligands, amines, peptide fragments, antibodies, and aptamers. In some embodiments, the vitamin is folic acid.

In some embodiments, the composition is lyophilized. In some embodiments, the polymeric nanoparticle has an average diameter of about 40-150 nm.

In some embodiments, the composition further comprises a tri-block copolymer of PEG-PPG-PEG. In some embodiments, the tri-block copolymer of PEG-PPG-PEG comprises poloxamer 407. In some embodiments, the tri-block copolymer of PEG-PPG-PEG comprises poloxamer 181.

The present disclosure further provides a method for preparing biodegradable polymeric nanoparticles comprising: (a) dissolving L-lactide, a polymer comprising methoxy-PEG and a block copolymer comprising PEG-PPG-PEG in an organic solvent to obtain a solution; (b) adding a Sn-catalyst to the solution to obtain a reaction mixture; (c) stirring the reaction mixture to obtain a hybrid block copolymer of PLA chemically modified with a block copolymer or polymer; (d) dissolving the block copolymer from step c in an organic solvent and homogenizing to obtain a homogenized mixture; (e) adding the homogenized mixture to an aqueous phase to obtain an emulsion; and (f) stirring the emulsion to obtain biodegradable polymeric nanoparticles; wherein the L-lactide undergoes ring opening polymerization. In some embodiments, the method optionally comprises the steps of washing the biodegradable polymeric nanoparticles with water and drying the biodegradable polymeric nanoparticles. In some embodiments, the biodegradable polymeric nanoparticles have an average diameter in the range of about 40-150 nm. In some embodiments, step (a) optionally comprises adding emulsifier. In some embodiments, at least steps (e) and (f) are performed in a batch process. In some embodiments, at least steps (e) and (f) are performed in a continuous process. In some embodiments, the Sn-catalyst is stannous octoate. In some embodiments, the stannous octoate is added in amount of about 0.005% by weight, based on the total weight of the reaction mixture. In some embodiments, step (b) further comprises adding a base.

The present disclosure also provides a method for preparing biodegradable polymeric nanoparticles comprising: (a) dissolving a m-PEG-PLA block copolymer and a PLA-PEG-PPG-PEG-PLA penta-block copolymer in a first organic solvent to obtain an organic solution; (b) adding the organic solution to an aqueous phase to obtain an emulsion; and (c) stirring the emulsion to obtain biodegradable polymeric nanoparticles. In some embodiments, the method further comprises, before steps (a)-(c): dissolving L-lactide, a polymer comprising m-PEG, and a block copolymer comprising PEG-PPG-PEG in a second organic solvent and adding a Sn-catalyst to obtain a reaction mixture; and stirring the reaction mixture to obtain a m-PEG-PLA and PLA-PEG-PPG-PEG-PLA. In some embodiments, the Sn-catalyst is stannous octoate. In some embodiments, the stannous octoate is added in amount of about 0.005% by weight, based on the total weight of the reaction mixture. In some embodiments, at least steps (a) and (b) are performed in a batch process. In some embodiments, at least steps (a) and (b) are performed in a continuous process. In some embodiments, the aqueous phase comprises an emulsifier. In some embodiments, the organic phase further comprises a therapeutic agent. In some embodiments, the therapeutic agent comprises salinomycin. In some embodiments, step (b) comprises continuously adding the aqueous phase and the organic phase to a container, thereby obtaining the emulsion. In some embodiments, the organic phase further comprises a therapeutic agent, and the organic phase is provided as a single stream comprising the m-PEG-PLA block copolymer, the PLA-PEG-PPG-PEG-PLA block copolymer, and the salinomycin. In some embodiments, the organic phase further comprises a therapeutic agent, and the organic phase is provided as a two streams, with a first organic stream comprising the m-PEG-PLA block copolymer and the PLA-PEG-PPG-PEG-PLA block copolymer, and a second organic stream comprising the salinomycin. In some embodiments, the aqueous phase comprises an emulsifier. In some embodiments, the emulsifier comprises poloxamer 407. In some embodiments, the emulsifier comprises poloxamer 181.

### BRIEF DESCRIPTION OF THE DRAWINGS

Aspects, features, benefits, and advantages of the embodiments described herein will be apparent with regard to the following description, appended claims, and accompanying drawings. The drawings depict exemplary embodiments of the disclosure and are not intended to be limiting.

FIG. 1 illustrates the reaction scheme of ring opening polymerization of L-lactide.

FIG. 2A shows the FTIR spectrum of PLA based hybrid block copolymer—m-PEG-PLA+PLA-PEG-PPG-PEG-PLA. FIG. 2B shows proton NMR of PLA based hybrid block copolymer—m-PEG-PLA+PLA-PEG-PPG-PEG-PLA.

FIG. 3 shows a particle diameter distribution of salinomycin loaded nanoparticles.

FIG. 4 shows a release profile of salinomycin by salinomycin loaded nanoparticles in PBS at 37° C.

FIG. 5 shows that internalization of the hybrid PLA nanoparticles was confirmed by the release of rhodamine inside cytoplasm in (A) MCF-7 cells, (B) MDA-MB 231 cells, and (C) MDA-MB 468 cells.

FIG. 6 shows percent inhibition of cell proliferation in (A) MCF-7, (B) MDA-MB 468, and (C) MDA-MB 231 cell lines, compared to free salinomycin.

FIG. 7 shows dose response of free salinomycin and salinomycin loaded nanoparticles in BALB/c mice when administered intraperitoneally.

FIG. 8 shows dose response of free salinomycin and salinomycin loaded nanoparticles in BALB/c mice when administered intravenously.

FIG. 9 shows elution of salinomycin as observed at 205 nm in Xterra MS C18 column in the peak at 11.25 min (A) and calibration curve of salinomycin plotted as a function of area under peak (B).

FIG. 10 shows schematic diagram of continuous preparation of biodegradable polymeric nanoparticles

FIG. 11 shows proton NMR of block copolymers (di-block and penta-block) were dissolved at 10 mg/mL in deuterated chloroform (CDCl₃) mixed at 85:15 di-block to penta-block ratio.

FIG. 12 shows comparison of drug-loaded nanoparticles generated by batch-wise vs continuous in-flow wise preparation.

FIG. 13 shows stability in 50% glucose at 2-8 C of salinomycin drug-loaded nanoparticles.

FIG. 14 shows an exemplary batch process for producing polymeric nanoparticles of the present disclosure.

FIG. 15 shows a first exemplary continuous process for producing polymeric nanoparticles of the present disclosure.

FIG. 16 shows a second exemplary continuous process for producing polymeric nanoparticles of the present disclosure.

FIG. 17 shows the chemical structure of salinomycin.

### DETAILED DESCRIPTION

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise. As used in the specification and the appended claims, the singular forms “a,” “an” and “the” include plural referents unless the context clearly dictates otherwise. Unless otherwise indicated, conventional methods of mass spectroscopy, NMR, HPLC, protein chemistry, biochemistry, recombinant DNA techniques and pharmacology are employed. The use of “or” or “and” means “and/or” unless stated otherwise. Furthermore, use of the term “including” as well as other forms, such as “include”, “includes,” and “included,” is not limiting. As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least.” When used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The section headings used herein are for organizational purposes only and are not to be construed as limiting the subject matter described.

As used herein, “consisting of” and grammatical equivalent thereof exclude any element, step or ingredient not specified in the claim.

As used herein, the term “about” or “approximately” means within 5% of a given value or range.

The term “biodegradable” as used herein refers to both enzymatic and non-enzymatic breakdown or degradation of the polymeric structure in an organism or other biological system.

The term “cationic” refers to any agent, composition, molecule or material that has a net positive charge or positive zeta potential under the respective environmental conditions. In various embodiments, nanoparticles described herein include a cationic polymer, peptide, protein carrier, or lipid. As used herein, the term “multi-drug resistant” refers to cancer cells that have developed resistance to two or more chemotherapy drugs. Cancer cells can become multi-drug resistant by multiple mechanisms including decreased drug uptake and increased drug efflux.

As used herein, the term “resistant” or “refractive” to a therapeutic agent when referring to a cancer patient means that the cancer has innate, or achieved resistance to, the effects of the therapeutic agent as a result of contact with the therapeutic agent. Stated alternatively, the cancer is resistant to the ordinary standard of care associated with the particular therapeutic agent.

As used herein, the term “nanoparticle” refers to particles in the range between 10 nm to 1000 nm in diameter, wherein diameter refers to the diameter of a perfect sphere having the same volume as the particle. The term “nanoparticle” is used interchangeably as “nanoparticle(s)”. In some cases, the diameter of the nanoparticle is in the range of about 1-1000 nm, 10-500 nm, 20-300 nm, or 100-300 nm. In various embodiments, the diameter is about 30-170 nm. In certain embodiments, the diameter of the nanoparticle is about 1, 5, 10, 25, 50, 75, 100, 125, 150, 175, 200, 225, 250, 275, 300, 325, 350, 375,400, 425, 450, 475, 500, 525, 550, 575, 600, 625, 650, 675, 700, 725, 750, 775, 800, 825, 850, 875, 900, 925, 950, 975, or 1000 nm. In other embodiments, the diameter of the nanoparticle is 1, 5, 10, 25, 50, 75, 100, 125, 150, 175, 200, 225, 250, 275, 300, 325, 350, 375,400, 425, 450, 475, 500, 525, 550, 575, 600, 625, 650, 675, 700, 725, 750, 775, 800, 825, 850, 875, 900, 925, 950, 975, or 1000 nm.

In some cases, a population of particles may be present. As used herein, the diameter of the nanoparticles is an average of a particular population of nanoparticles.

As used herein, the term “polymer” is given its ordinary meaning as used in the art, i.e., a molecular structure comprising one or more repeat units (monomers), connected by covalent bonds. The repeat units may all be identical, or in some cases, there may be more than one type of repeat unit present within the polymer. When more than one type of repeat unit in present in a single polymer, the polymer is termed a “copolymer” or “co-polymer.” As used herein, a “block copolymer” is a copolymer formed when repeat units cluster together and form groups (“blocks”) of repeating units. As used herein, a “hybrid” block copolymer comprises a mixture of different block copolymers, such as the m-PEG-PLA block copolymer and PLA-PEG-PPG-PEG-PLA block copolymer described herein.

As used herein, the term “associated substantially with” in the context of a nanoparticle means a substance is encapsulated by the nanoparticle, adsorbed to the nanoparticle, or conjugated to a surface of the nanoparticle. In some embodiments, when a substance is associated substantially with a nanoparticle, at least 80%, at least 90%, at least 95%, or at least 99% of the mass of the substance is encapsulated by the nanoparticle, adsorbed to the nanoparticle, or conjugated to the surface of the nanoparticle.

As used herein, an “emulsifier” and “emulsion” are given their ordinary meaning as used in the art. That is, an emulsion is a chemical mixture comprising a dispersed phase and a continuous phases, wherein the phases are normally immiscible. An emulsifier can stabilize the components of an emulsion such that the kinetic stability of the emulsion is increased. Examples of emulsifiers that may optionally be included in a composition with the polymeric nanoparticles of the present disclosure include: PEG-PPG-PEG of different molecular weights from 1,000 Da to 13,000 Da such as, for example, from 4,000 Da to 13,000 Da or from 1,000 Da to 6,000 Da and sodium lauryl sulphate. An emulsifier may or may not be added to the polymeric nanoparticles of the present disclosure (e.g., may or may not be added during preparation thereof). The emulsifier may be a polymeric emulsifier (e.g., the PEG-PPG-PEG tri block copolymer). The emulsifier may be a non-polymeric emulsifier (e.g., sodium lauryl sulfate). Polymeric and non-polymeric emulsifiers may be used alone, in combination, or not at all.

A “chemotherapeutic agent,” “therapeutic agent,” or “drug” is a biological (large molecule) or chemical (small molecule) compound useful in the treatment of cancer, regardless of mechanism of action. Large, biological molecules are polymers or oligomers and include proteins, peptides, and nucleic acids. Small, chemical molecules are not polymers and include therapeutic molecules such as alkylating agents and antibiotics. “Low molecular weight,” compounds, as used herein, are not polymers or oligomers and have a molecular weight of less than about 5,000 g/mol. Classes of chemotherapeutic agents include, but are not limited to: alkylating agents, antimetabolites, spindle poison plant alkaloids, cytotoxic/antitumor antibiotics, topoisomerase inhibitors, proteins, peptides, antibodies, photosensitizers, and kinase inhibitors. Chemotherapeutic agents include compounds used in “targeted therapy” and non-targeted, conventional chemotherapy. A “targeting moiety” is a molecule that will bind selectively to the surface of targeted cells. For example, the targeting moiety may be a ligand that binds to the cell surface receptor found on a particular type of cell or expressed at a higher frequency on target cells than on other cells.

The targeting moiety or therapeutic agent can be a peptide or protein. “Proteins” and “peptides” are well-known terms in the art, and as used herein, these terms are given their ordinary meaning in the art. Generally, peptides are amino acid sequences of less than about 100 amino acids in length, and proteins are generally considered to be molecules of at least 100 amino acids. The amino acids can be in D- or L-configuration. A protein can be, for example, a protein drug, an antibody, a recombinant antibody, a recombinant protein, an enzyme, or the like. In some cases, one or more of the amino acids of the peptide or protein can be modified, for example by the addition of a chemical entity such as a carbohydrate group, a phosphate group, a farnesyl group, an isofamesyl group, a fatty acid group, a linker for conjugation, functionalization, or other modification such as cyclization, by-cyclization and any of numerous other modifications intended to confer more advantageous properties on peptides and proteins. In other instances, one or more of the amino acids of the peptide or protein can be modified by substitution with one or more non-naturally occurring amino acids. The peptides or proteins may by selected from a combinatorial library such as a phage library, a yeast library, or an in vitro combinatorial library.

The term “combination,” “therapeutic combination,” or “pharmaceutical combination” as used herein refer to the combined administration of two or more therapeutic agents (e g., co-delivery). Components of a combination therapy may be administered simultaneously or sequentially, i.e., at least one component of the combination is administered at a time temporally distinct from the other component(s). In embodiments, a component(s) is administered within one month, one week, 1-6 days, 18, 12, 10, 9, 8, 7, 6, 5, 4, 3, 2, 1 hour, or 30, 20, 15, 10, or 5 minutes of the other component(s).

The term “pharmaceutically acceptable” as used herein refers to those compounds, materials, compositions and/or dosage forms, which are, within the scope of sound medical judgment, suitable for contact with the tissues a warm-blooded animal, e.g., a mammal or human, without excessive toxicity, irritation allergic response and other problem complications commensurate with a reasonable benefit/risk ratio.

A “therapeutically effective amount” of a polymeric nanoparticle comprising one or more therapeutic agents is an amount sufficient to provide an observable or clinically significant improvement over the baseline clinically observable signs and symptoms of the disorders treated with the combination.

The term “subject” or “patient” as used herein is intended to include animals, which are capable of suffering from or afflicted with a cancer or any disorder involving, directly or indirectly, a cancer. Examples of subjects include mammals, e.g., humans, apes, monkeys, dogs, cows, horses, pigs, sheep, goats, cats, mice, rabbits, rats, and transgenic non-human animals. In an embodiment, the subject is a human, e.g., a human suffering from cancer.

The term “treating” or ‘treatment” as used herein comprises a treatment relieving, reducing or alleviating at least one symptom in a subject or producing a delay in the progression of a disease. For example, treatment can be the diminishment of one or several symptoms of a disorder or complete eradication of a disorder, such as cancer. Within the meaning of the present disclosure, the term “treat” also denotes to arrest and/or reduce the risk of worsening a disease. The term “prevent”, “preventing” or “prevention” as used herein comprises the prevention of at least one symptom associated with or caused by the state, disease or disorder being prevented.

As used herein, the term “human equivalent dose” refers to a dose of a composition to be administered to a human that is calculated from a specific dose used in an animal study.

As used herein, the term “rapidly proliferating cells” refers to cells having the capacity for autonomous growth (e.g., cancer cells).

As used herein, the term “cancer stem cell” refers to a cancer cell that has characteristics of a stem cell, such as giving rise to all cell types within a particular tumor type and the ability to self-renew. In some embodiments, the cancer stem cell is resistant or refractory to chemotherapy.

As used herein, “contrast agents” and “imaging agents” are chemical substances that can be introduced into a biological sample or organism to be imaged. These agents can improve or change the imaging characteristics of the tissues to be imaged. In some embodiments, these agents can be delivered by the polymeric nanoparticles of the present disclosure. Examples these agents include iodine-based agents, barium sulfate, and gadolinium.

As used herein, a “stabilizer” reduces or eliminates changes in diameter and/or PDI of polymeric nanoparticles during storage or lyophilization. Examples of stabilizers that can be used with the polymeric nanoparticles of the present disclosure include: mannose, beta-lactose, trehalose, sodium cholate, and glucose. When a stabilizer is employed, it may be present in a weight of about 5% to about 50% of the total weight of the polymer such as, for example, 10% to 50%, 30% to 50%, or 40% to 50% of the total weight of the polymer. In some embodiments, the stabilizer can comprise glucose.

As used herein, “protein transduction domains” are small peptides able to carry proteins, peptides, nucleic acid, nanoparticles, and viral particles, across cellular membranes and into cells. These domains can optionally be linked (e.g., via a covalent bond) to an anti-cancer peptide of the present disclosure. When used in this manner, the polyarginine domain can comprise 8-10 arginine residues such as, for example, nine arginine residues.

As used herein, one g/mole is equivalent to one “Dalton” (i.e., Dalton and g/mol are interchangeable when referring to the molecular weight of a polymer). “Kilodalton” as used herein refers to 1,000 Daltons.

### Polymeric Nanoparticles

Polymeric nanoparticles of the present disclosure can comprise one or more polylactide block copolymers. The polylactide is made by the process of subjecting a compound to chemical reaction, wherein the compound may be lactide, glycolide, or a mixture thereof. The reaction may be between (1) the lactide, glycolide, or mixture thereof and (2) PEG and/or PEG-PPG-PEG of different molecular weights ranging from 2,000 Da to 14,000 Da, mPEG, or other initiators described below. The chemical reaction may be a ring opening polymerization (ROP) (Dechy-Cabaret et al., 2004, Chem. Rev., 104, 12, 6147-6176; Kricheldorf et al., 1995, Polymer., 36, 6, 1253-1259; Schwach et al., 1997, Polym Sci A: Polym Chem 35: 3431-3440; Degee et al., 1999, Marcomolecular Symposia, 144, 1, 289-302; Ryner et al., 2001, Macromolecules, 34, 12, 3877-3881). An organic catalyst or an inorganic catalyst may be used in said ROP. The organic catalyst may be tin(II) 2-ethylhexanoate (also called stannous octoate and abbreviated as Sn(Oct)2), tin(II)trifluoromethane sulfonate (Sn(OTf)2), 4-(dimethylamino)pyridine (DMAP). In certain embodiments, an alcohol initiator is used in the ROP. The alcohol initiator may be benzyl alcohol, methoxy-poly(ethylene glycol) (mPEG), 1,1,1-tris(hydroxymethyl)ethane (TE), pentaerythritol (PE), a poloxamer (such as poloxamer 181 (e.g., PLURONIC® L-61), poloxamer 407 (e.g., PLURONIC® F127)), or any other multi-hydroxy compound. Additional examples of poloxamers that may be used as initiators in the present disclosure include poloxamer 101, poloxamer 108, poloxamer 124, poloxamer 188, poloxamer 184, poloxamer 182, poloxamer 237, and poloxamer 338.

In an embodiment, the polymeric nanoparticles provided herein comprise a block copolymer comprising poly(lactic acid) (PLA) and polyethylene glycol) (PEG). Poly(lactic acid) (PLA), is a hydrophobic polymer, and is a polymer appropriate for inclusion in the polymeric nanoparticles.

PEG is another suitable component of the polymer used to form the polymeric nanoparticles as it imparts hydrophilicity, anti-phagocytosis against macrophages, and resistance to immunological recognition. Block copolymers like polyethylene glycol)-poly(propylene glycol)-poly(ethylene glycol) (PEG-PPG-PEG) are hydrophilic or hydrophilic-hydrophobic copolymers that can be used in the present invention. Block copolymers may have two, three, four, five, or more numbers of distinct blocks.

In a further embodiment, the polymeric nanoparticles provided herein comprise methoxy-poly(ethylene glycol)-poly(lactic acid) (m-PEG-PLA) di-block copolymer.

In some embodiments, the polymeric nanoparticles can comprise a di-block copolymer methoxy-poly(ethylene glycol)-poly(lactic acid) (m-PEG-PLA) and a penta-block copolymer poly(lactic acid)-poly(ethylene glycol)-poly(propylene glycol)-poly(ethylene glycol)-poly(lactic acid) (PLA-PEG-PPG-PEG-PLA). The di-block and penta-block copolymers may both be generated from a ROP reaction as described above and in the Examples.

In some embodiments, the di-block copolymer comprises an average molecular weight of about 5,000 to 30,000 g/mol.

In some embodiments, the penta-block copolymer comprises an average molecular weight of about 5,000 to 30,000 g/mol.

In some embodiments, the polymeric nanoparticles are biodegradable.

In a further embodiment, the polymeric nanoparticles provided herein comprise poly(lactic acid)-poly(ethylene glycol) (PLA-PEG) di-block copolymer.

In yet a further embodiment, the polymeric nanoparticles provided herein comprise poly(lactic acid)-poly(ethylene glycol)-poly(propylene glycol)-poly(ethylene glycol) (PLA-PEG-PPG-PEG) tetra-block copolymer. In various embodiments, the nanoparticles comprise a NANOPRO™, which is a biodegradable, long blood circulating, stealth, tetra-block polymeric nanoparticle platform (NanoProteagen; Massachusetts). The PLA-PEG-PPG-PEG tetra-block copolymer can be formed from chemical conjugation of PEG-PPG-PEG triblock copolymer with PLA. The PLA-PEG-PPG-PEG tetra-block copolymer is described in U.S. Pat. No. 10,092,617, which is incorporated herein by reference in its entirety.

In some embodiments, a tri-block copolymer of PEG-PPG-PEG may be included in a composition comprising the nanoparticles disclosed herein. The PEG-PPG-PEG copolymer can function as a cryoprotectant and/or emulsifier. The inventors of the present disclosure have found that including such a triblock copolymer can improve the quality of the polymeric nanoparticles after freezing and/or can serve as an emulsifier. In some embodiments, the triblock copolymer may be associated with or associated substantially with the polymeric nanoparticles. In some embodiments, the triblock copolymer may not be associated with the polymeric nanoparticles. In some embodiments, the triblock copolymer may not be associated substantially with the polymeric nanoparticles. In some embodiments, the triblock copolymer comprises a central poly(propylene glycol) block flanked by two poly(ethylene glycol) blocks. In some embodiments, the triblock copolymer comprises poloxamer 407. In some embodiments, the triblock copolymer comprises poloxamer 181. In other embodiments, the emulsifier (e.g., tri-block copolymer) is used in an amount of about 0.5% to 5% by weight, based on the total weight of the composition. In some embodiments, an emulsifier may not be included.

Targeted delivery of the polymeric nanoparticles loaded with therapeutic agents can be achieved, compared to free drug formulations (e.g., formulations of drugs without nanoparticles). The nanoparticles of the present invention can also be surface conjugated, bioconjugated, or adsorbed with one or more entities including targeting moieties on the surface of nanoparticles. Targeting moieties can cause nanoparticles to localize onto a tumor, disease site, site of interest for imaging, or any other desired site and release a therapeutic agent at that location. The targeting moiety can bind to or associate with linker molecules. Targeting molecules include but are not limited to antibody molecules, growth receptor ligands, vitamins, peptides, haptens, aptamers, and other targeting molecules known to those skilled in the art. Drug molecules and imaging molecules can also be attached to the targeting moieties on the surface of the nanoparticles directly or via linker molecules.

Specific, non-limiting examples of targeting moieties include vitamins, ligands, amines, peptide fragments, antibodies, aptamers, a transferrin, an antibody or fragment thereof, sialyl Lewis X antigen, hyaluronic acid, mannose derivatives, glucose derivatives, cell specific lectins, galaptin, galectin, lactosylceramide, a steroid derivative, an RGD sequence, EGF, EGF-binding peptide, urokinase receptor binding peptide, a thrombospondin-derived peptide, an albumin derivative and/or a molecule derived from combinatorial chemistry.

### Therapeutic Agents and Pharmaceutical Compositions

In some embodiments, the biodegradable polymeric nanoparticle further comprises a therapeutic agent. For example, the therapeutic agent can be associated with the polymeric nanoparticles by being contained within an enclosed region of a shell of polymer. Alternatively or additionally, the therapeutic agents can be interspersed within the polymer that forms the shell, or the therapeutic agents can adsorb to an outside surface of the shell. The therapeutic agents can be associated with the polymeric nanoparticle in any manner suitable to carry and deliver the therapeutic agents to locations of disease in need of treatment.

In certain embodiments, the therapeutic agent can be associated substantially with the polymeric nanoparticles. In some embodiments, the same polymeric nanoparticles can comprise more than one therapeutic agent. In some embodiments, the therapeutic agent(s) are encapsulated by the nanoparticle.

In some embodiments, the therapeutic agent is selected from a group comprising small organic molecules, nucleic acids, polynucleotides, oligonucleotides, nucleosides, DNA, RNA, amino acids, peptides, proteins, antibiotics, chemotherapeutics, drugs, metal ions, dyes, radioisotopes, contrast agents and imaging agents.

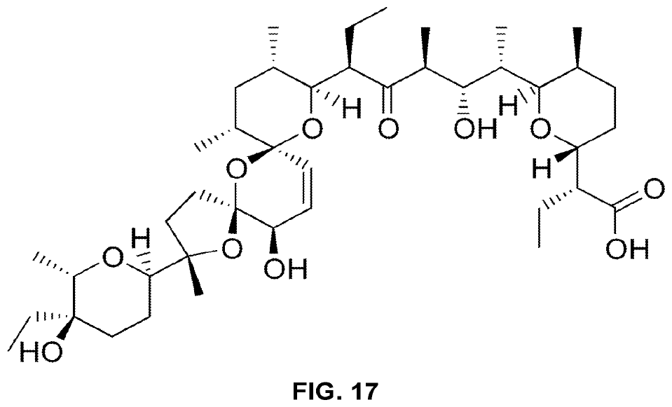

In some embodiments, the therapeutic agent comprises the antibiotic salinomycin. Salinomycin has the chemical structure shown in FIG. 17.

In some embodiments, the therapeutic agent comprises bortezomib.

In some embodiments the therapeutic agent comprises a therapeutic nucleic acid, protein, or peptide sequence. In some embodiments, the nucleic acid, protein, or peptide sequence comprises a DNA, RNA, or amino acid sequence selected from the groups of genes consisting of: insulin, interferon-γ (IFN-γ), tumor necrosis factor (TNF), and interleukin-2 (IL-2). In some embodiments, the DNA, RNA, or amino acid sequence is from the corresponding human gene or is a fragment thereof. Human sequences for these genes are accessible with the following NCBI reference sequence accession numbers: insulin (NP_000198.1 and NM_000207.3); IFN-γ (NP_000610.2 and NM_000619.3); TNF (NP_000585.2 and NM_000594.4); and IL-2 (NP_000577 and NM_000586.4).

The polymeric nanoparticles of the present disclosure may be formulated into a pharmaceutical composition for administration to cell, tissue, or organism. In some embodiments, the polymeric nanoparticles or pharmaceutical composition can be administered for the treatment of disease such as cancer. In some embodiments of the pharmaceutical composition for use, the cancer is selected from the group consisting of breast cancer, ovarian cancer, pancreatic cancer, leukemia, lymphoma, osteosarcoma, gastric cancer, prostate cancer, colon cancer, lung cancer, liver cancer, kidney cancer, head and neck cancer, and cervical cancer. In an embodiment, the cancer is metastatic.

In another embodiment, the pharmaceutical composition for use further comprises administering an additional anti-cancer therapy to the subject. In an embodiment of the pharmaceutical composition for use, the additional anti-cancer therapy is surgery, chemotherapy, radiation, hormone therapy, immunotherapy, or a combination thereof.

In some embodiments of the pharmaceutical composition for use, the cancer is resistant or refractory to a chemotherapeutic agent.

In certain embodiments of the pharmaceutical composition for use, the subject is a human.

In an embodiment of the pharmaceutical composition for use, the composition further comprises a second therapeutic agent or a targeted anti-cancer agent.

### Preparation of Polymeric Nanoparticles

Methods of preparing biodegradable polymeric nanoparticles are also provided herein. In general, the method can include formation of nanoparticles from block copolymers. The method can also include synthesis of the block copolymers (e.g., via a polymerization reaction disclosed herein). The method can comprise: (a) dissolving a m-PEG-PLA block copolymer and a PLA-PEG-PPG-PEG-PLA penta-block copolymer in a first organic solvent to obtain an organic solution; (b) adding the organic solution to an aqueous phase to obtain an emulsion; and (c) stirring (e.g., mixing, pouring, or pumping together the organic solution and the aqueous phase) the emulsion to obtain biodegradable polymeric nanoparticles.

In some embodiments, the method further comprises, before steps (a)-(c): dissolving L-lactide, a polymer comprising m-PEG, and a block copolymer comprising PEG-PPG-PEG in a second organic solvent and adding a Sn-catalyst to obtain a reaction mixture; and stirring the reaction mixture to obtain a m-PEG-PLA and PLA-PEG-PPG-PEG-PLA. The di-block and penta-block components can be synthesized in separate reactions (e.g., in different solutions) or in a single reaction (e.g., in the same solution). In some embodiments, the initiator for the di-block can comprise m-PEG (optionally with an average molecular weight of 5,000 Da). In some embodiments, the initiator for the penta-block can comprise poloxamer 181 (e.g., PLURONIC® L-61) or poloxamer 407 (PLURONIC® F-127). The m-PEG-PLA and PLA-PEG-PPG-PEG-PLA block copolymers can be synthesized separately from the nanoparticles themselves, and/or the block copolymers can be stored before being used to prepare the polymeric nanoparticles. In some embodiments, the synthesis is an ROP reaction. In some embodiments, the ROP reaction is catalyzed by a Sn-catalyst. In some embodiments, the Sn-catalyst comprises stannous octoate. In some embodiments, the Sn-catalyst is added in amount of about 0.005% by weight, based on the total weight of the reaction mixture. In some embodiments, the stannous octoate is added in amount of about 0.005% by weight, based on the total weight of the reaction mixture.

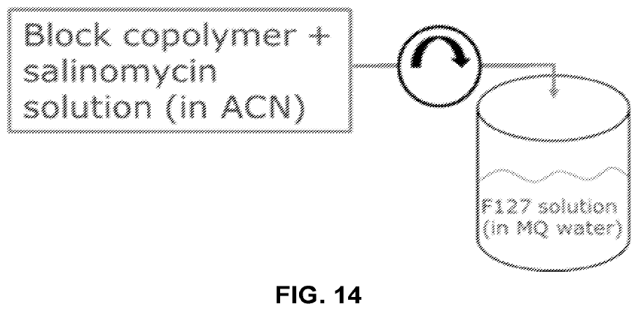

In some embodiments, at least steps (a) and (b) (and optionally (c)) are performed in a batch process (e.g., FIG. 14).

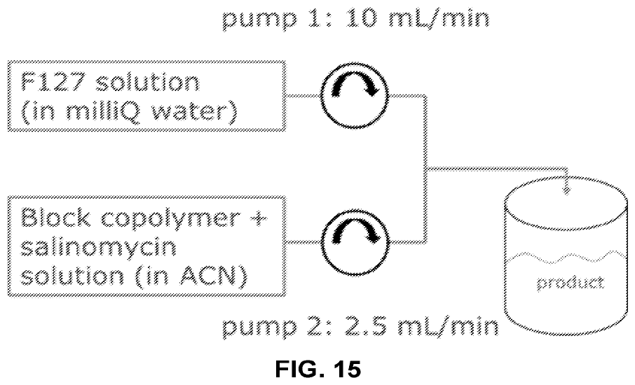

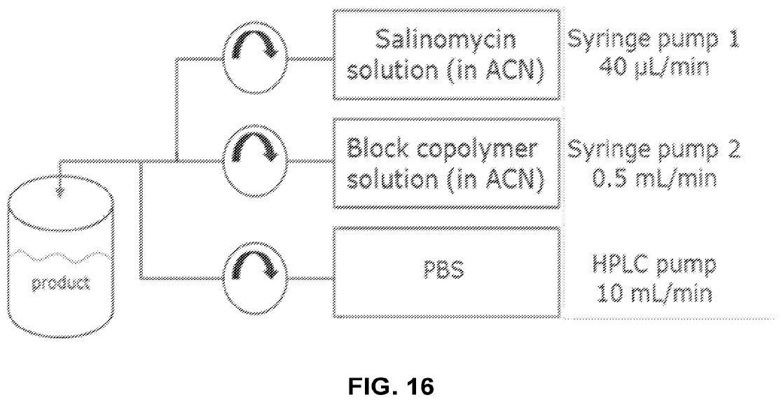

In some embodiments, at least steps (a) and (b) (and optionally (c)) are performed in a continuous process (e.g., FIG. 15 or 16). A continuous process can be beneficial by allowing for increased practicality and scale when preparing the polymeric nanoparticles.

In some embodiments, the aqueous solution comprises PBS (phosphate-buffered saline), purified water, and/or the tri-block PEG-PPG-PEG copolymer. In some embodiments, the tri-block PEG-PPG-PEG copolymer such as poloxamer 407 or 181.

In some embodiments, the organic phase further comprises a drug. In some embodiments, the drug comprises salinomycin. In some embodiments, the drug comprises bortezomib. In some embodiments, the drug comprises any drug disclosed herein, or a combination thereof.

In some embodiments, step (b) comprises continuously adding the aqueous phase and the organic phase to a container (e.g., the “product” container of FIGS. 15 and 16), thereby obtaining the emulsion.

When preparing a polymeric nanoparticles by a continuous or batch process, the various components described above may be combined in any suitable method to produce the polymeric nanoparticles with sufficient efficiency and quality. For example, when the organic phase further comprises a drug, the organic phase can be provided as two streams, with a first organic stream comprising the m-PEG-PLA block copolymer and the PLA-PEG-PPG-PEG-PLA block copolymer, and a second organic stream comprising the salinomycin (See FIG. 16). Alternatively, the organic phase may still comprise the drug, and the organic phase may be provided as a single stream comprising the m-PEG-PLA block copolymer, the PLA-PEG-PPG-PEG-PLA block copolymer, and the drug (See FIG. 15). The block copolymers may also be provided together in a single stream or separately in their own respective streams.

Regardless of the chemistry (e.g., ROP, condensation polymerization) and process (e.g., batch, continuous) used to prepare the biodegradable polymeric nanoparticles, and in some embodiments, a purification step may be performed after the formation of the polymeric nanoparticles. For example, the nanoparticles may be washed one or more times in order to remove unreacted or partially reacted components, catalyst, solvents, and any other undesired components. Alternatively or additionally, one or more precipitations can be performed in order to remove unreacted or partially reacted components, catalyst, solvents, and any other undesired components. Alternatively or additionally, tangential flow filtration (TFF) can be performed to purify the polymeric nanoparticles.

Regardless of the chemistry and process used to prepare the biodegradable polymeric nanoparticles, and in some embodiments, a filtration and/or sterilization step may be performed after the formation of the polymeric nanoparticles. For example, the nanoparticles may be filtered and/or sterilized by a 0.2 micron filter.

Those skilled in the art will be aware that the invention described herein is subject to variations and modifications other than those specifically described. It is to be understood that the invention described herein includes all such variations and modifications. The invention also includes all such steps, features, compositions and compounds referred to or indicated in this specification, individually or collectively, and any and all combinations of any two or more of the steps or features.

### EXAMPLES

The disclosure will now be illustrated with working examples, and which is intended to illustrate the working of disclosure and not intended to restrictively any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

### Example 1. Ring Opening Polymerization of PLA-PEG-PPG-PEG-PLA Penta-Block and Hybrid Block Copolymeric Nanoparticles

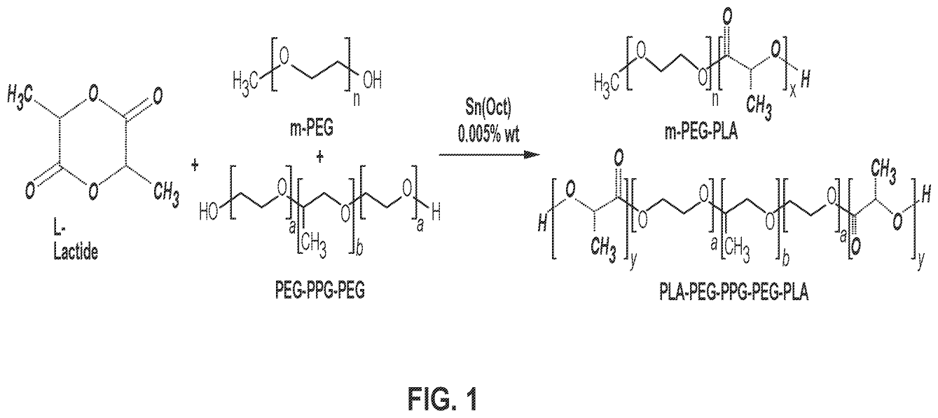

The mPEG-PLA+PLA-PEG-PPG-PEG-PLA (hybrid copolymer) and PLA-PEG-PPG-PEG-PLA (penta-block copolymer) were prepared by ring opening polymerization using stannous octoate. The scheme of the ring opening polymerization reaction is shown in FIG. 1.

A condensation polymerization reaction for the production of PLA-PEG-PPG-PEG-PLA comprised the following steps. 5 g of poly (lactic acid) (PLA) with an average molecular weight of 60,000 g/mol was dissolved in 100 ml CH₂Cl₂ (dichloromethane) in a 250 ml round bottom flask. To this solution, 0.7 g of PEG-PPG-PEG polymer (molecular weight range of 1100-8400 g/mol) was added. The solution was stirred for 10-12 hours at 0° C. To this reaction mixture, 5 ml of 1% N,N-dicyclohexylcarbodimide (DCC) solution was added followed by slow addition of 5 ml of 0.1% 4-Dimethylaminopyridine (DMAP) at −4° C. to 0° C./sub-zero temperatures. The reaction mixture was stirred for the next 24 hours followed by precipitation of the PLA-PEG-PPG-PEG block copolymer with diethyl ether and filtration using Whatman filter paper No. 1. The PLA-PEG-PPG-PEG block copolymer precipitates so obtained were dried under low vacuum and stored at 2° C. to 8° C. until further use.

In contrast, the ring opening polymerization comprised the following steps. First, a polymer comprising m-PEG, L-lactide, and a block co-polymer comprising PEG-PPG-PEG were dissolved in an organic solvent to obtain a solution. About 0.005% stannous octoate and a base were added to the solution to obtain a reaction mixture. The reaction mixture was stirred in presence of nitrogen and at 170° C. for 3 hours, to obtain a hybrid block copolymer of PLA, chemically modified with a block copolymer or polymer. The block polymer was dissolved in an organic solvent and made into a homogenized mixture. The homogenized mixture was added to an aqueous phase to obtain an emulsion. Finally, the emulsion was stirred to obtain biodegradable polymeric nanoparticles, to promote L-lactide to undergo ring opening polymerization.

The advantages of adopting the ring opening polymerization over the condensation polymerization are summarized in Table 1.

TABLE 1
Advantages of ring opening polymerization.

| Ring Opening Polymerization | Condensation Polymerization |
|---|---|
| 1. One pot synthesis, 3-5 hours | 1. 2 pot synthesis, 48 hours |
| reaction time and high yield | reaction time and lesser yield |
| (85-90%). | (50-55%). |
| 2. Catalyst used is stannous | 2. Catalyst used is DMAP/DCC |
| octoate FDA approved. | not approved by FDA and may |
| | have toxic issues |
| 3. Narrow molecular weight | 3. Broad molecular weight |
| distribution with PDI of 1.1-1.2 | distribution with PDI of 1.3-1.6 |
| 4. Nanoparticle diameter is | 4. Nanoparticle diameter is 70-110 |
| 50-70 nanometers with lesser | nanometers with broad PDI 0.06-0.3 |
| PDI 0.01-0.03 | |

### Example 2. Characterization of PLA-PEG-PPG-PEG-PLA Penta-Block and Hybrid Block Copolymeric Nanoparticles

We compared blank hybrid mPEG-PLA+PLA-PEG-PPG-PEG-PLA, synthesized by ring opening polymerization versus penta-block PLA-PEG-PPG-PEG-PLA polymer based on yield, monomer to initiator mole ratio, nanoparticle diameter and stability. The results are shown in Table 2. It was observed that the hybrid blank mPEG-PLA+PLA-PEG-PPG-PEG-PLA at [M]/[I] ratio of 133 and blank PLA-PEG-PPG-PEG-PLA at [M]/[I] ratio of 126 were the most stable.

TABLE 2
Characterization of blank PLA based block copolymeric nanoparticles (NPs)

| | Monomer | | | | |
|---|---|---|---|---|---|
| | (Lactide) to | Polymer's | | NP^{b}s avg | |
| | Initiator | Mw (GPC^{a}) | Polymer's | diameter | NPs |
| Polymer | Mole Ratio | g/mol | Yield % | (nm) | Stability |
| mPEG-PLA + | 16.5 | 5632 | 52 ± 3 | 36 ± 5 | Not |
| PLA-PEG-PPG- | | | | | Stable |
| PEG-PLA (ROP) | | | | | |
| (Hybrid) | | | | | |
| mPEG-PLA + | 33.25 | 7860 | 69 ± 5 | 42 ± 3 | Not |
| PLA-PEG-PPG- | | | | | Stable |
| PEG-PLA (ROP) | | | | | |
| (Hybrid) | | | | | |
| mPEG-PLA + | 51 | 10908 | 70 ± 6 | 48 ± 3 | Not |
| PLA-PEG-PPG- | | | | | Stable |
| PEG-PLA (ROP) | | | | | |
| (Hybrid) | | | | | |
| mPEG-PLA + | 133 | 25302 | 85 ± 5 | 64 ± 5 | Stable |
| PLA-PEG-PPG- | | | | | |
| PEG-PLA (ROP) | | | | | |
| (Hybrid) | | | | | |
| PLA-PEG-PPG- | 126 | 26503 | 84 ± 5 | 87 ± 2 | Stable |
| PEG-PLA (ROP) | | | | | |
| (Penta-block) | | | | | |

^{a}gel permeation chromatography
^{b}nanoparticle

When salinomycin was incorporated into the hybrid and the penta-block nanoparticles, the hybrid nanoparticle was found to be more stable. The results of the characterization of salinomycin loaded nanoparticle are shown in Table 3, before and after lyophilization. Table 4 summarizes the advantages of hybrid nanoparticles over penta-block nanoparticles.

TABLE 3
Characterization of Salinomycin loaded PLA based Penta-
block and Hybrid Block Copolymeric Nanoparticles

| | | | Before | | | |
|---|---|---|---|---|---|---|
| | | | lyophilization | After lyophilization | | Drug |
| | Synthesis | | Diameter/ | Diameter/ | Zeta | loading % |
| Polymer | route | EE % | PDI^{c} | PDI | potential | (wt/wt) |
| PLA-PEG-PPG- | Ring opening | 71 ± 3% | 93 ± 4 nm/ | 1003 ± 86 nm/ | −8.14 ± 1.8 | 2.75 ± 0.09% |
| PEG-PLA (ROP) | polymerization | | 0.23 ± 0.02 | 0.25 ± 0.04 | | |
| (Penta-block) | of L-Lactide | | | (Unstable) | | |
| mPEG-PLLA + | Ring opening | 73 ± 2% | 70 ± 5 nm/ | 78 ± 4 nm/ | −15.3 ± 1.5 | 2.85 ± 0.07% |
| PLLA-PEG-PPG- | polymerization | | 0.15 ± 0.03 | 0.13 ± 0.03 | | |
| PEG-PLLA | of L-Lactide | | | (Stable) | | |
| (Hybrid) | | | | | | |

^{c}polydispersity index

TABLE 4
Advantage of hybrid over penta-block copolymeric nanoparticles

| Hybrid Block Copolymeric NPs | Penta-block Copolymeric NPs |
|---|---|
| 1. Nanoparticle diameter is | 1. Nanoparticle diameter is 80-110 |
| 50-70 nanometers with lesser | nanometers with broader PDI 0.2-0.5 |
| PDI 0.01-0.03 | |
| 2. Salinomycin loaded NPs are | 2. Salinomycin loaded NPs are not |
| stable after lyophilization | stable after lyophilization with |
| with diameter 75-85 nm | diameter >1 micron |

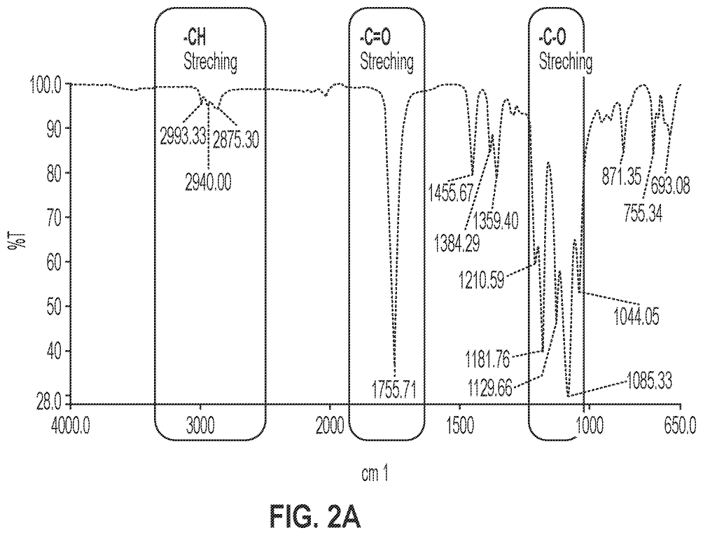

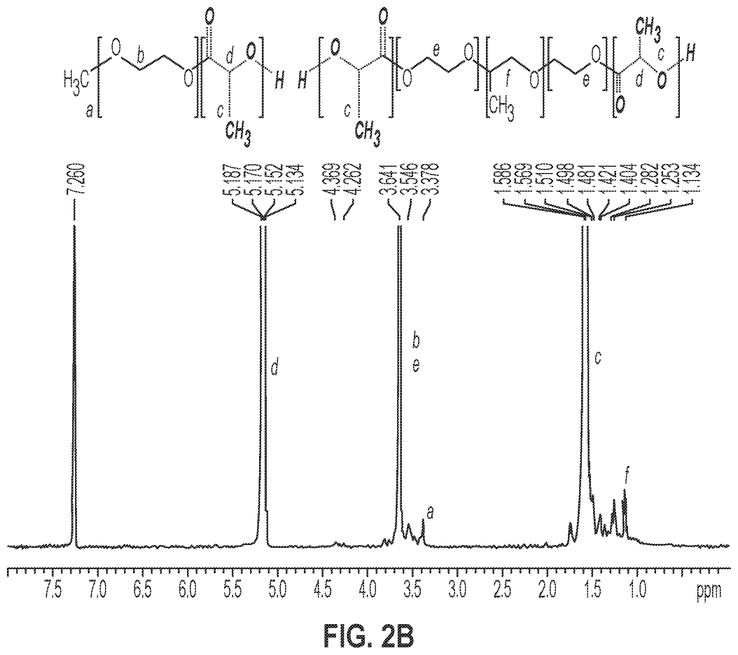

FIG. 2A and FIG. 2B show the FTIR and proton NMR spectrum of PLA based hybrid block copolymer—m-PEG-PLA+PLA-PEG-PPG-PEG-PLA respectively.

Next, various reagents were probed for the cryoprotection of salinomycin-loaded PLA based hybrid block copolymeric nanoparticles. The summary of the reagents probed and they effect on the salinomycin nanoparticle after lyophilization are detailed in Table 5. The lowest polydispersity index and nanoparticle diameter was observed with glucose.

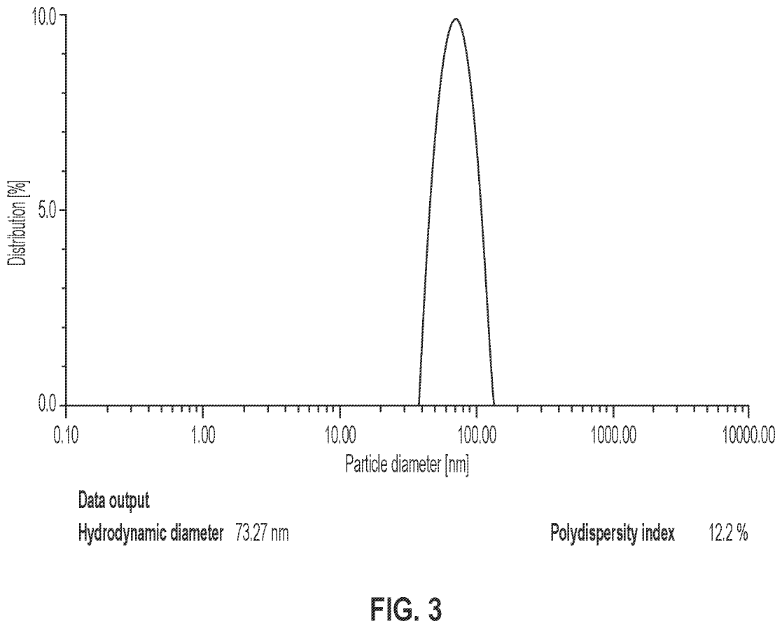

Further, the concentration of glucose was optimized to a value that yielded the lowest polydispersity and diameter. Table 6 summarizes the diameter and polydispersity index for various concentrations of glucose. It was observed that 50% glucose yielded the lowest values. FIG. 3 shows the particle diameter distribution of salinomycin loaded nanoparticles. They hydrodynamic diameter of the salinomycin loaded PLA based hybrid block copolymeric nanoparticles was 73.27 at a PDI of 12.2%.

TABLE 5
Selection of agents for stabilization of SAL-loaded
PLA based hybrid block copolymeric nanoparticles

| | Diameter before | Diameter after |
|---|---|---|
| Cryoprotectant | lyophilization/PDI | lyophilization/PDI |
| None | 74.38 nm/0.17 | 1470 nm |
| Mannose | | 158.31 nm/0.264 (Micron |
| | | particles also seen) |
| Beta-lactose | | 85.35 nm/0.249(Micron |
| | | particles also seen) |
| Trehalose | | 80.36 nm/0.186 |
| Sodium cholate | | 82 nm/0.134 |
| Glucose | | 79.39 nm/0.11 |

TABLE 6
Concentration of glucose optimization for stabilization of
SAL-loaded PLA based hybrid block copolymeric nanoparticles

| Before | | | | |
|---|---|---|---|---|
| lyophilization | After lyophilization | | | |
| — | Cryoprotectant (Polymer weight %) | | | |
| — | 5% glucose | 15% glucose | 30% glucose | 50% glucose |
| Diameter/ | Diameter/ | Diameter/ | Diameter/ | Diameter/ |
| PDI | PDI | PDI | PDI | PDI |
| 72.2 nm/0.16 | 145.9 nm/ | 129.7 nm/ | 133.3 nm/ | 79.3 nm/ |
| | 0.25 + | 0.22 + | 0.22, But | 0.14 Highly |
| | Micron | Micron | settle | stable NPs |
| | particles | particles | with time. | |
| | also seen | also seen | | |

### Example 3. Release of Salinomycin from SAL-Loaded PLA Based Hybrid Block Copolymeric Nanoparticles In Vitro, Ex Vivo and In Vivo

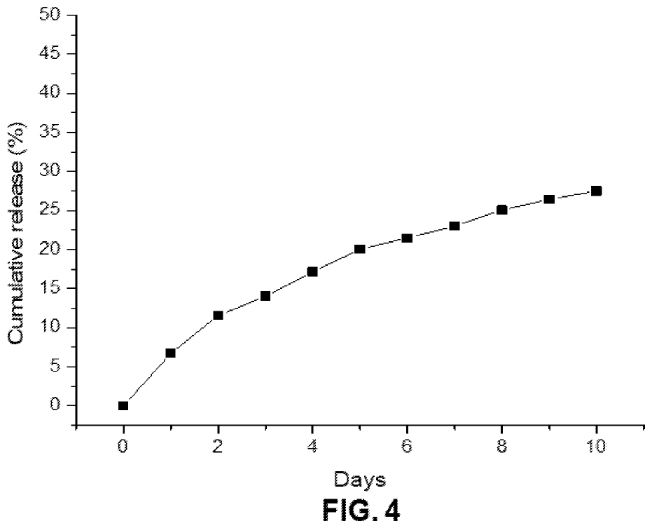

The release of salinomycin from nanoparticles in phosphate buffered saline (PBS), pH 7.4, was probed over 10 days at 37° C. The release profile of salinomycin in this in vitro setting is shown in FIG. 4. Cumulative release of SAL from nanoparticles in 10 days was approximately 27% of total encapsulated SAL concentration.

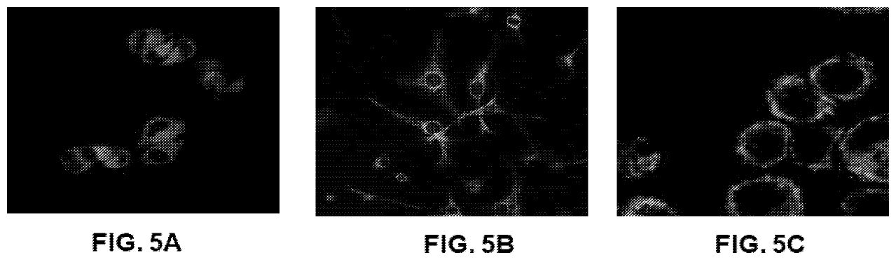

Next, the internalization of rhodamine 123 loaded hybrid PLA nanoparticles in MCF-7, MDA-MB 231 and MDA-MB 468 cell lines was probed using confocal microscopy. The internalization of the hybrid PLA nanoparticles was confirmed by the release of rhodamine inside cytoplasm in each cell line as seen in FIG. 5.

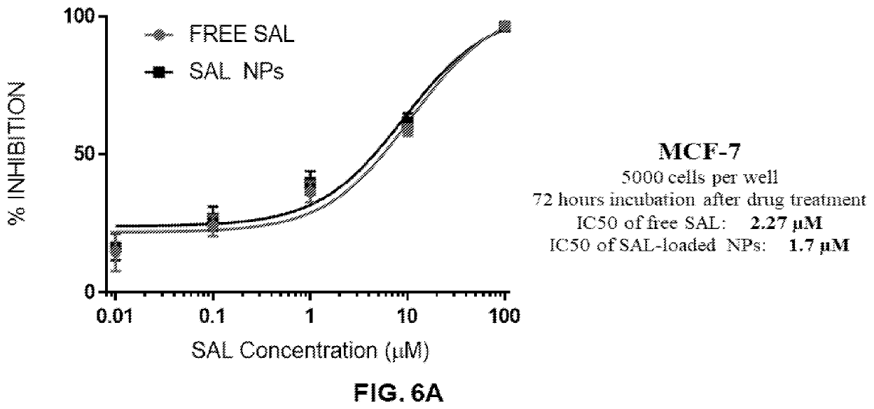

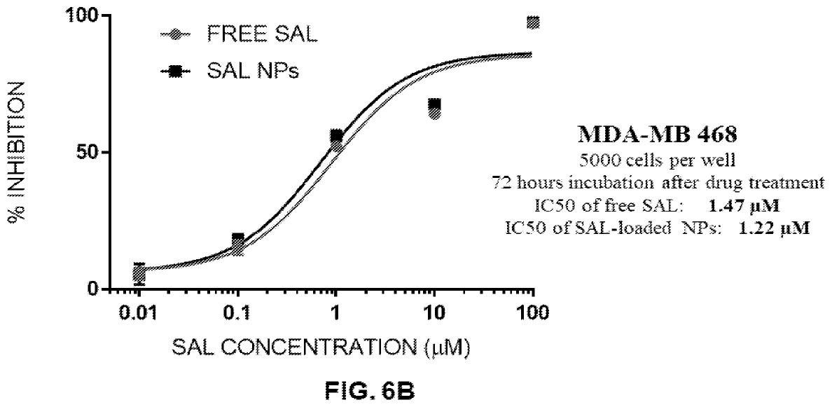

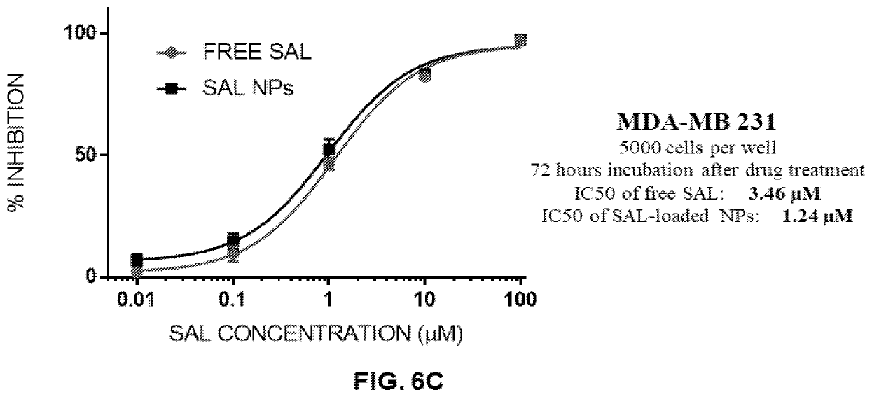

Next, the potency of inhibition of salinomycin-induced cell growth was probed by comparing the inhibition with salinomycin and salinomycin nanoparticle in MCF-7, MDA-MB 231 and MDA-MB 468 cell lines. It was observed that the salinomycin loaded nanoparticle was as potent as free salinomycin in inhibition of cell growth. (see FIG. 6A, B, C). MCF-7 cells at concentration of 5000 cells per well were treated with salinomycin or salinomycin nanoparticles from a concentration of 0.01-100 μM respectively for 72 hours. The IC50 for MCF-7 cells with free salinomycin was 2.27 μM and with salinomycin loaded nanoparticles was 1.7 μM. MDA-MB 468 cells at concentration of 5000 cells per well were treated with salinomycin or salinomycin nanoparticles from a concentration of 0.01-100 μM respectively for 72 hours. The IC50 for MDA-MB 468 cells with free salinomycin was 1.47 μM and with salinomycin loaded nanoparticles was 1.22 μM. MDA-MB 231 cells at concentration of 5000 cells per well were treated with salinomycin or salinomycin nanoparticles from a concentration of 0.01-100 μM respectively for 72 hours. The IC50 for MDA-MB 231 cells with free salinomycin was 3.46 μM and with salinomycin loaded nanoparticles was 1.24 μM.

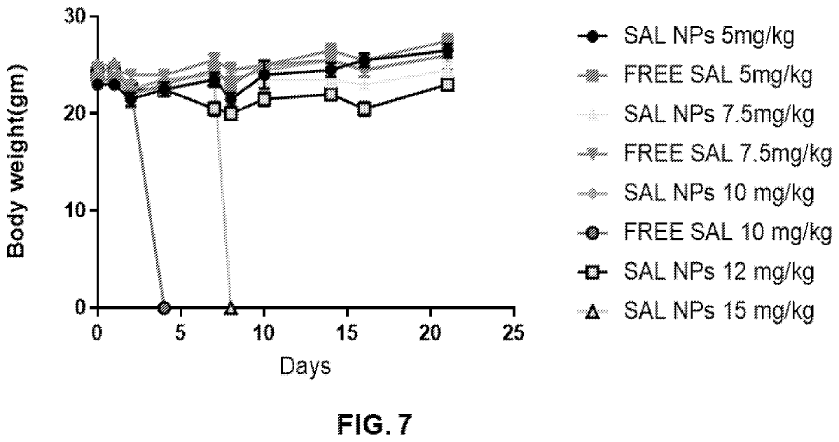

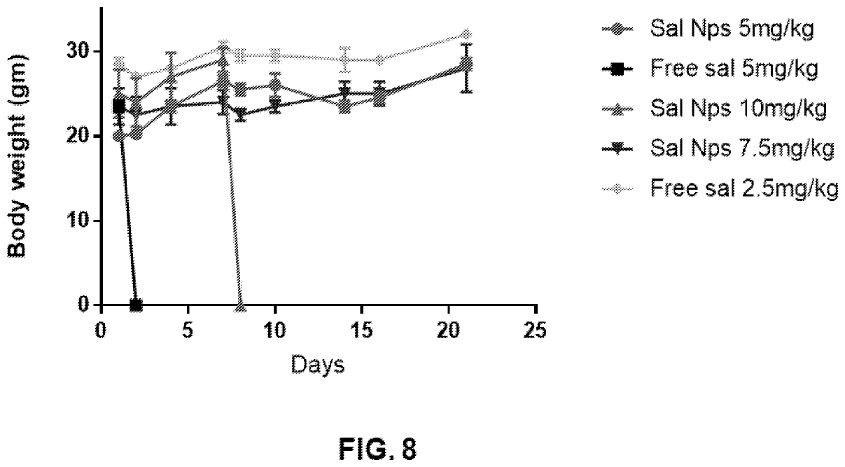

Next, the maximum tolerated dose of salinomycin and salinomycin-loaded PLA based hybrid block copolymeric salinomycin nanoparticle was probed in BALB/c mice. Mice were administered the salinomycin loaded hybrid block copolymeric nanoparticles once a week for 3 weeks. The body weight reduction was observed as a phenotypic response. The dose-response of salinomycin and the salinomycin nanoparticle was observed by administration intraperitoneally (FIG. 7) and intravenously (FIG. 8). For intraperitoneal administration, mice were administered salinomycin and salinomycin loaded nanoparticles at 5 mg/kg, 7.5 mg/kg, 10 mg/kg. Further, only the salinomycin nanoparticles were administered at 12 mg/kg and 15 mg/kg, intraperitoneally. Three mice were probed at each dose for salinomycin and salinomycin loaded nanoparticle. For intravenous administration, mice were administered salinomycin and salinomycin loaded nanoparticles at 5 mg/kg. Only salinomycin nanoparticles were administered at 1 mg/kg, and 7.5 mg/kg. Further another group of salinomycin at 2.5 mg/kg was also tested. Three mice were probed at each dose for salinomycin and salinomycin loaded nanoparticle. It was observed that free salinomycin was tolerated up to a dose of 10 mg/kg and the salinomycin loaded nanoparticle was tolerated up to 15 mg/kg, when administered intraperitoneally. When administered intravenously, free salinomycin was lethal at 5 mg/kg, while the salinomycin nanoparticles were tolerated up to 10 mg/kg.

### Example 4. Determination of Salinomycin Content

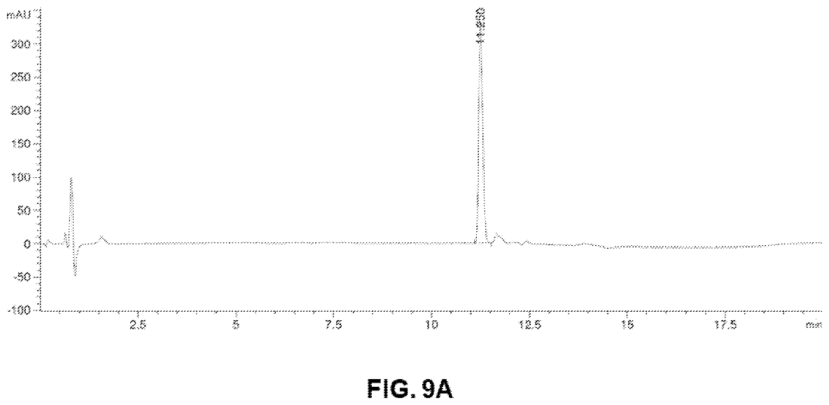

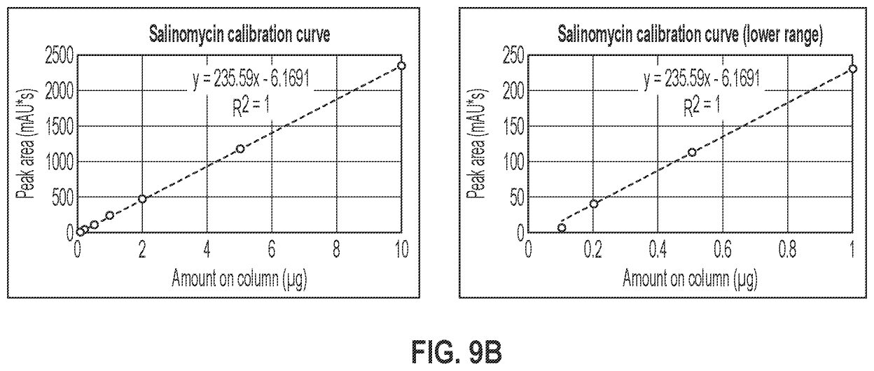

The amount of salinomycin was calculated using HPLC, by loading 10 μl salinomycin in a C18 column (Xterra MS C18) with a 205 nm detector, eluted with methanol/water/acetic acid mixture at 0.35 mL/min. The area under the peak at 11.25 min was observed and the concentration was calculated using the calibration chart provided in FIG. 9.

### Example 5. Scale-Up Manufacturing Process—Continuous Preparation of Biodegradable Polymeric Nanoparticles

A schematic diagram of an exemplary scale up process is shown in FIG. 10. Tri-block PEG-PPG-PEG (as PLURONIC® F127 and/or PLURONIC® L-61) was delivered in the aqueous stream in purified water (e.g., Mili-Q water) at 10 ml/min. Poly-L-lactide block copolymers (m-PEG-PLA and PLA-PEG-PPG-PEG-PLA) in acetonitrile (ACN) were delivered at 2.5 ml/min. It is noted that “block copolymer” and “block copolymer solution” as used in FIGS. 10 and 14-16 refer the “hybrid block copolymer” mixture comprising the di- and penta-block copolymers.

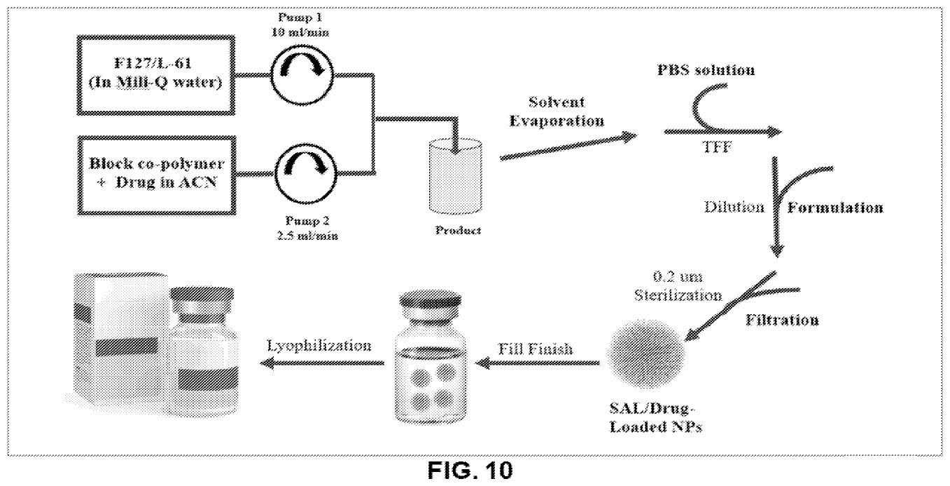

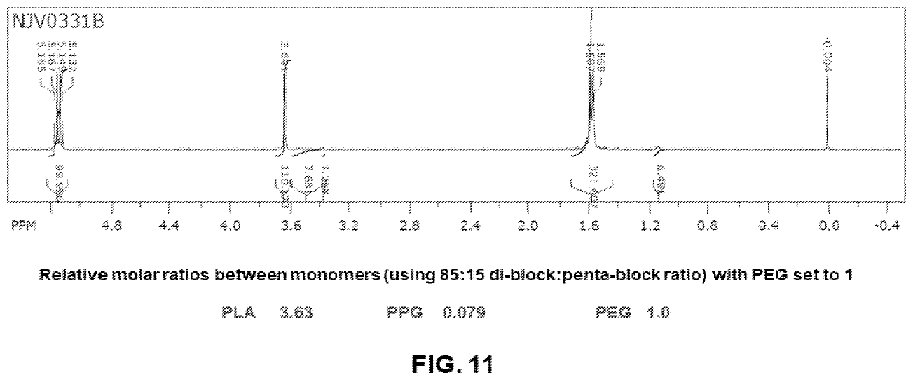

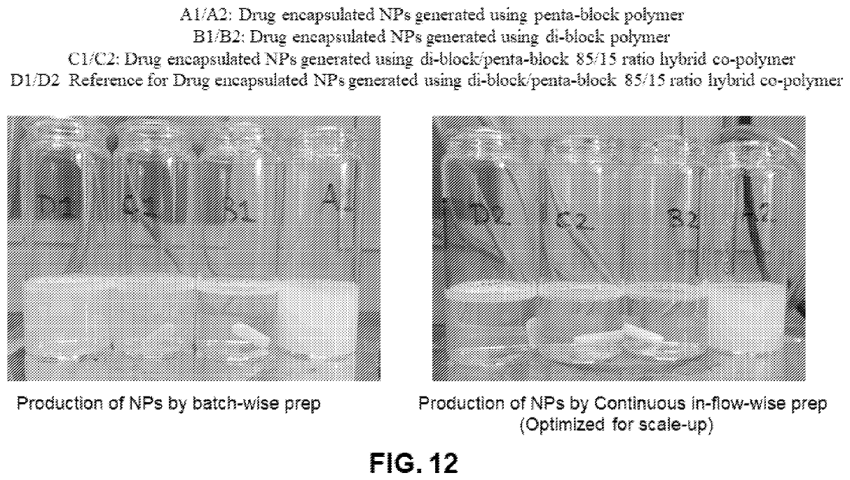

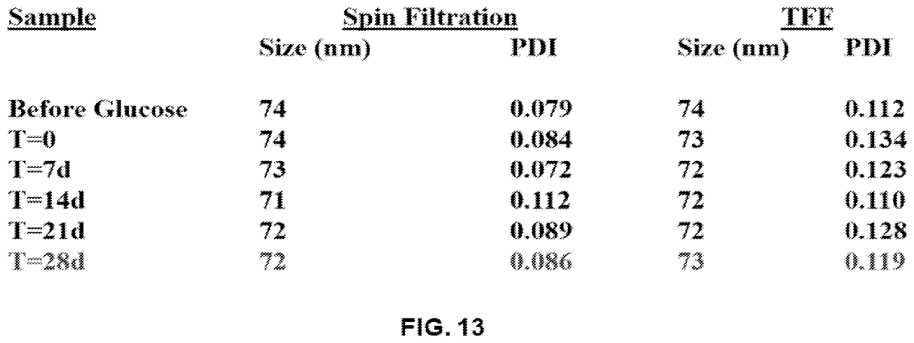

The two streams were combined in a container to allow for nanoparticle formation. The contents of the container were concentrated by solvent evaporation. PBS was added to the loaded nanoparticles, which were purified using tangential flow filtration (TFF). For example, 42.5 mL of nanoparticle solution obtained after solvent evaporation was filtered over 0.22 μm polyethersulfone (PES) membrane and diluted to 80 ml in a single step. Nanoparticle solution was loaded onto 3 kDa TFF membrane nanoparticle solution and was concentrated to ˜20 ml (=1 dia-filtration volume DV). Nanoparticles were rinsed with 5 DVs water. Finally, the nanoparticle solution was rinsed off membrane and filtered over 0.22 μm PES membrane again. The nanoparticles were then diluted and formulated to a desired formulation. The formulated nanoparticles were sterilized using a 0.22 micron filter and filled in ampules. The nanoparticle formulation was finally lyophilized. The block copolymers (di-block and penta-block) were dissolved at 10 mg/mL in deuterated chloroform (CDCl₃), then mixed in 85:15 ratio and were analyzed by proton NMR (FIG. 11). It was observed that the nanoparticles generated using continuous in-flow-wise preparation had better polydispersity index and diameter, compared to a batch-wise preparation of nanoparticles (FIG. 12). Further, the final drug-loaded nanoparticles were observed to be stable at 2-8° C. in the presence of glucose for 28 days (FIG. 13).

The PLURONIC® F127 comprises a triblock copolymer comprising a central poly(propylene glycol) block flanked by two poly(ethylene glycol) blocks (e.g., poloxamer 407). The approximate lengths of the two PEG blocks can be 101 repeat units for each block, while the approximate length of the propylene glycol block can be 56 repeat units. PLURONIC® F127 is available from BASF SE, Ludwigshafen, Germany.

### Example 6. Synthesis of Di-Block and Penta-Block Copolymers and Scale-Up Manufacturing Process for Polymeric Nanoparticles

Di-block and penta-block copolymers can serve as starting materials for the manufacture of polymeric nanoparticles of the present disclosure. Exemplary methods that were performed to prepare these are provided below.

The penta-block copolymer is synthesized from initiator PLURONIC® L-61. PLURONIC® L-61 comprises polyoxypropylene with a molecular mass of 1800 g/mol and comprises a 10% polyoxyethylene content (poloxamer 181). PLURONIC® L-61 is available from BASF SE, Ludwigshafen, Germany. The di-block copolymer is synthesized from initiator mPEG5000 (Poly(ethylene glycol) methyl ether average Mn 5,000). Both syntheses occur by ring-opening polymerization of lactide in the presence of a Sn-catalyst (e.g., stannous octoate) with tetraglyme (Tetraethylene glycol dimethyl ether) as the solvent. Purification is performed through multiple rounds of precipitation to remove the catalyst, solvent, and lactic acid impurities to yield both polymers that are released according to the release criteria separately. The polymers may be stored as starting materials for manufacturing polymeric nanoparticles.

The manufacture process of salinomycin-loaded NPs (SAL-NPs) continues by dissolving both polymers in the appropriate ratio in acetonitrile at 60° C. to ensure full solvation. Once dissolved, the solution is cooled to 40° C. and used to prepare SAL-NPs.

Initially, batch wise and continuous flow setup were compared. Polymeric nanoparticles were first assembled through precipitation by controlled injection of a concentrated acetonitrile solution into an aqueous solution as a batch process (FIG. 14). The polymeric blend was dissolved in acetonitrile (20 mg/ml), heated to 60° C. to completely dissolve the polymer and cooled down to room temperature. Salinomycin was added from a 50 mg/ml solution in a 25:1 ratio (polymer:salinomycin) and the organic solution was then injected with a syringe pump into water with 5 mg/mL cryoprotectant PLURONIC® F127. Typically, SAL-NPs of ˜100 nm were spontaneously formed with a SAL content of ˜0.3 mg/ml.

A continuous flow setup was, subsequently, designed based on the batch process as for scalability and reproducibility (FIG. 15). Two HPLC pumps were employed to combine the organic and aqueous flow in a T-joint to allow for the continuous manufacturing of SAL-NPs. This strategy afforded comparable SAL-NP solutions as the batch process in terms of NP diameter and salinomycin content.

Moreover, to optimize the continuous flow set up for generating SAL-NPs, a continuous flow setup is employed which includes three pumps and two T-joints to combine an organic and aqueous solution that induces nanoparticle formation (FIG. 16). The warm polymeric solution is combined in the first T-joint with salinomycin dissolved in acetonitrile. Subsequently, the mixed acetonitrile solution is combined with an aqueous solution in a second T-joint, which induces the formation of polymeric nanoparticles loaded with salinomycin (SAL-NPs). Optionally, the aqueous solution can comprise PBS and/or a tri-block copolymer as emulsifier and/or cryoprotectant.

1. A composition comprising a biodegradable polymeric nanoparticle formed of hybrid block copolymers comprising a di-block copolymer methoxy-poly(ethylene glycol)-poly(lactic acid) (m-PEG-PLA) and/or a penta-block copolymer poly(lactic acid)-poly(ethylene glycol)-poly(propylene glycol)-poly(ethylene glycol)-poly(lactic acid) (PLA-PEG-PPG-PEG-PLA).

2. The composition of claim 1, wherein one or both of the di-block copolymer and the penta-block copolymer comprise an average molecular weight of about 5,000 to 30,000 g/mol and/or wherein the polymeric nanoparticle has an average diameter of about 40-150 nm.

3. (canceled)

4. The composition of claim 1, wherein the composition is substantially free of emulsifier or wherein the composition further comprises external emulsifier of about 0.5% to 5% by weight.

5. (canceled)

6. The composition of claim 1, wherein the biodegradable polymeric nanoparticle further comprises a therapeutic agent, optionally wherein the therapeutic agent is associated substantially with the biodegradable polymeric nanoparticle.

7. (canceled)

8. The composition of claim 76, wherein the therapeutic agent is selected from a group comprising small organic molecules, nucleic acids, polynucleotides, oligonucleotides, nucleosides, DNA, RNA, amino acids, peptides, proteins, antibiotics, low molecular weight molecules, chemotherapeutics, drugs, metal ions, dyes, radioisotopes, contrast agents and imaging agents, optionally comprising a stabilizer.

9. The composition of claim 8, wherein the antibiotic is salinomycin.

10-13. (canceled)

14. The composition of claim 8, wherein the peptide is an anti-cancer peptide.

15. The composition of claim 14, wherein the anticancer peptide is either FSRSLHSLL (SEQ ID NO: 1) or any polypeptide substantially incorporating the FSRSLHSLL (SEQ ID NO: 1), and wherein the FSRSLHSLL (SEQ ID NO: 1) is in either the D or L-configuration.

16-17. (canceled)

18. The composition of claim 14, wherein the anti-cancer peptide is CQCRRKN (SEQ ID NO: 2), a sequence from the MUC1-CD domain, or wherein the anti-cancer peptide is AQARRKN (SEQ ID NO: 3), a modified sequence from the MUC1-CD domain, optionally wherein the anticancer peptide is linked to a protein transduction domain, further optionally wherein the protein transduction domain comprises a polyarginine domain.

19-22. (canceled)

23. The composition of claim 228, wherein the chemotherapeutic is selected from the group consisting of paclitaxel, doxorubicin, pimozide, perimethamine, topoisomerase I inhibitors such as irinotecan, topotecan, indenoisoquinolines, or nor-indenoisoquinolines, HDAC6 inhibitors, PI3Kalpha, beta, gamma or delta inhibitors or PI3-Kdelta/HDAC6 dual inhibitors.

24. The composition of claim 8, wherein the therapeutic agent is DNA, optionally wherein the DNA comprises a polynucleotide encoding a protein comprising the amino acid sequence of TNF, IL-2 or gamma Interferon.

25-26. (canceled)

27. The composition of claim 1, wherein the biodegradable polymeric nanoparticle further comprises a targeting moiety selected from the group consisting of vitamins, small molecule drugs, ligands, amines, peptide fragments, antibodies, and aptamers.

28. (canceled)

29. A lyophilized composition of claim 1.

30. (canceled)

31. The composition of claim 1, further comprising a tri-block copolymer of PEG-PPG-PEG, optionally wherein the tri-block copolymer of PEG-PPG-PEG comprises poloxamer 407 or poloxamer 181.

32-33. (canceled)

34. A method for preparing biodegradable polymeric nanoparticles comprising:
(a) dissolving L-lactide, a polymer comprising methoxy-PEG and a block copolymer comprising PEG-PPG-PEG in an organic solvent to obtain a solution;
(b) adding a Sn-catalyst to the solution to obtain a reaction mixture;
(c) stirring the reaction mixture to obtain a hybrid block copolymer of PLA chemically modified with a block copolymer or polymer;
(d) dissolving the PLA-modified block copolymer or polymer from step c in an organic solvent and homogenizing to obtain a homogenized mixture;
(e) adding the homogenized mixture to an aqueous phase to obtain an emulsion; and
(f) stirring the emulsion to obtain biodegradable polymeric nanoparticles;
wherein the L-lactide undergoes ring opening polymerization.

35. The method of claim 34, wherein said method optionally comprises the steps of washing the biodegradable polymeric nanoparticles with water and drying the biodegradable polymeric nanoparticles, and/or wherein the biodegradable polymeric nanoparticles have a diameter in the range of about 40-150 nm, and/or wherein step (a) optionally comprises adding emulsifier, and/or wherein at least steps (e) and (f) are performed in a batch process, and/or wherein at least steps (e) and (f) are performed in a continuous process.

36-39. (canceled)

40. The method of claim 34, wherein the Sn-catalyst is stannous octoate, optionally wherein the stannous octoate is added in amount of about 0.005% by weight, based on the total weight of the reaction mixture.

41-42. (canceled)

43. A method for preparing biodegradable polymeric nanoparticles comprising:
(a) dissolving a m-PEG-PLA block copolymer and a PLA-PEG-PPG-PEG-PLA penta-block copolymer in a first organic solvent to obtain an organic solution;
(b) adding the organic solution to an aqueous phase to obtain an emulsion; and
(c) stirring the emulsion to obtain biodegradable polymeric nanoparticles.

44. The method of claim 43, further comprising, before steps (a)-(c):
dissolving L-lactide, a polymer comprising m-PEG, and a block copolymer comprising PEG-PPG-PEG in a second organic solvent and adding a Sn-catalyst to obtain a reaction mixture; and
stirring the reaction mixture to obtain a m-PEG-PLA and PLA-PEG-PPG-PEG-PLA and/or wherein the Sn-catalyst is stannous octoate, optionally wherein the stannous octoate is added in amount of about 0.005% by weight, based on the total weight of the reaction mixture.

45-46. (canceled)

47. The method of claim 43, wherein at least steps (a) and (b) are performed in a batch process or in a continuous process, optionally wherein the aqueous phase comprises an emulsifier and/or wherein the organic phase further comprises a therapeutic agent.

48-52. (canceled)

53. The method of claim 47, wherein the organic phase further comprises a therapeutic agent, and wherein the organic phase is provided as a single stream comprising the m-PEG-PLA block copolymer, the PLA-PEG-PPG-PEG-PLA block copolymer, and the therapeutic agent or wherein the organic phase further comprises a therapeutic agent, and wherein the organic phase is provided as a two streams, with a first organic stream comprising the m-PEG-PLA block copolymer and the PLA-PEG-PPG-PEG-PLA block copolymer, and a second organic stream comprising the therapeutic agent.

54-57. (canceled)
